(19) United States
(12) Patent Application Publication
(10) Pub. No.: US 2006/0293198 A1
(43) Pub. Date: Dec. 28, 2006

(54) Film-shape soap

(76) Inventor: Vanessa Rosa del Sol Constain Ordonez, Bogota. D. C. (CO)

Correspondence Address: HECTOR M. REYES RIVERA, Suite 1, 1060 Ashford Ave., San Juan, PR, 00907, US

(21) Appl. No.: 11/166,445

(22) Filed: Jun. 24, 2005

(51) Int. Cl. A61K 8/02

(52) U.S. Cl. 510/141

(57) ABSTRACT

The instant invention relates to a film-shape soap comprising a natural polymer and the method of its preparation thereof. More particularly the invention relates to film-shape soap and its preparation thereof wherein a natural polymer derivative of d-galacturonic acid is used and wherein no synthetic polymers are involved.

### BACKGROUND OF THE INVENTION

The instant invention relates to a film-shape soap comprising a polymer and the method of its preparation thereof. More particularly the invention relates to film-shape soap and its preparation thereof wherein a natural polymer derivative of d-galacturonic acid is used and wherein no synthetic polymers are involved.

Polymer containing film-shape soaps are known in the art. For instance, Carbonnel in Spanish patent 356,001 discloses a film shape soap comprising carboxymethyl cellulose and a copolymer derivative of polyvinyl acetate.

On the other hand, Nagarajan et al U.S. Pat. No. 4,828,752 discloses a toilet soap containing a polymeric thickener. Nagarajan's soap comprises a solid soap in different forms-such as bar, flakes, etc. Nagarajan's method of preparation of the said soap requires synthetic polymers and polyamides, preferably acrylic acids and alkyl acrylate derivatives as the thickeners agents.

Similarly Fujita et al, U.S. Pat. No. 5,062,986 discloses film-shape soap comprising a water soluble polymer. Particularly, Fujita discloses a polymer that is a polyester derivative of polyether polyol that can be prepared by the addition of an alkylkene oxide to a compound having two active hydrogen atoms in a molecule.

Interestingly, Fujita points out that while the demand of a film-shape soap has become increased for portable use, disposable use, unit use and so on, the intends to improve the quality of the same by incorporating various kind of a film formable polymer into the soap have been failed due to the incompatibility of the soap with the conventional film formable polymer material and the high molecular weight of the soap itself. All of the above cited documents are herein incorporated by reference.

Thus there is a need for a film-shape soap prepared by an economical method and wherein the obtained product is flexible, totally water soluble, portable, disposable and convenient to carry and used in a variety of sanitary circumstances.

### SUMMARY OF THE INVENTION

The instant invention relates to a film-shape soap highly convenient for personal, potable used as well as for a unit use comprising a natural water soluble polymer derivative of D-galacturonic acid having an average molecular weight of 20,000 to near 400,000 and soap.

The obtained film shape soap is totally soluble in water, highly flexible, provides cleanliness, softness, and skin moisturizing. It is highly convenient to use since it totally disintegrates into foam upon used, leaving no annoying residues.

The instant invention also provides a convenient method for the preparation of the said soap by mixing aqueous solutions of soap with aqueous solutions of the natural polymer derivative of D-galacturonic acid.

In addition to the excellent qualities of the obtained product, the instant method of preparation of the film shape soap is time and energy saving, environment-friendly and economical since the required polymer is already available from nature thus no synthetic polymers are prepared or required in the preparation of the instant film-shape soap.

### DETAILED DESCRIPTION OF THE INVENTION

The instant invention relates to a film-shape soap that does not contain synthetic polymers and that comprises a natural water soluble polymer derivative of D-galacturonic acid having a molecular weight from 20,000 to 400,000 and soap.

The only required polymer of the instant invention is a natural polymer derivative of D-galacturonic acid also known as pectin. It is well known in the chemistry arts and it is a lineal polymer highly soluble in water and partially esterified with methyl groups. It is present in cell walls of fruits and plants and commonly used in the manufacture of jam and jellies.

The said polymer can be extracted by a variety of methods well known in the art, see for instance, Morris T. N. Principles of Fruit Preservation-Jam Making, Canning and Drying, Volume VI, chapter 1, pages 11-27 (1933); McKee, U.S. Pat. No. 1,380,572; Doell et al, U.S. Pat. No. 1,385,525 and Jameson et al, and U.S. Pat. No. 1,497,884 wherein different extractions procedures of the said polymer are described. All of the above cited documents are herein incorporated by reference.

Thus, the natural polymer is readily available since it may be obtained directly from its natural source and is further used in the preparation of the instant invention. The availability of the said polymer and the incorporation of the said natural compound into the soap process preparation are excellent advantages over previous film-shape soap preparation methods since less energy and time as well as less money and efforts are required in the said preparation.

The required soap is not a limiting factor in the instant invention since it may be any type of soap. As it is well known in the chemical arts, a soap is an alkaline base of a fatty acid or a fatty oil

That may be prepared by conventional soap preparation methods including the saponification of animal or vegetable fatty acids or oils.

The phrase-alkali salt of the fatty acid-includes any kind of soap such as body soap, toilet soap, deodorant soap, medical soap, and the likes.

The said soap may be prepared by any conventional method, such as the saponification of a fatty acid or oil, for instance it includes saturated or unsaturated fats and oils. Preferably fatty acids or oils that are included in the invention are those commonly used in the soap industry, for example aranquinodic, esteric, lauric, linoleic, linolenic, oleic, palmitic and myristic acids palm oil, olive oil, coconut oil, soy oil, castor oil and similar ones.

Similarly, the alkali base required for the saponification process is any commonly alkaline base conventionally used in the soap preparation method and does not represent a limiting factor in the instant invention. Nonetheless, preferably alkali bases are those having sodium or potassium as the alkaline metal cations.

Optionally substances commonly used in the soap industry as additives may be added to the film-shape soap described herein. Among others, those selected from one or more of the group comprising fragrances, color agents, moisturizers, antioxidants, antibiotic agents and other similar derivatives.

The film shape soap has a thickness of from about 5 micrometers to one millimeter and more preferable from 20 micrometer to about 300 micrometers. The preparation of the film shape soap requires the initial preparation of aqueous solutions of the natural polymer derivative of D-galacturonic acid having a preferably concentration of 6-12% by weight and aqueous soap solutions having a preferably concentration of 16-29% by weight.

The said solutions may contain other additives commonly added to soaps such as fragrances, color agents, moisturizers, antibiotic agents and antioxidants among others. Both solutions are prepared with continuous stirring and maintaining the temperature range in about 0-50° C. Once prepared, the said solutions are mixed. The said mixing may be done by adding the aqueous alkali salt solution to aqueous solution containing the polymer or vice versa. The mixing is carried out with continuous stirring and while the temperature is less or equal to that 50° C.

The pH of the said solution is adjusted by addition of a base in such a way that it is higher than the pH of the aqueous alkali salt solution prepared at the beginning of the process.

The final mixture is then allowed to dry by extending the same on a flat anti-adhesive surface.

The instant invention also embraces other embodiments of the process resulting from variations in the sequence of the steps in carrying the described process. For instance adding the soap additives to the solution obtained after the mixing of the soap solution and the polymer containing solution and previously drying it or by adding the said additives to any one of the solutions that are mixed, for instance, the soap solution or the polymer containing solution or even by adding the said additives to both of the said solutions.

The following examples are presented to illustrate the invention. They are not construed in any way to limit the invention to the particular procedures indicated in the said examples.

### EXAMPLE 1

A solution of 20 ml of 6.5% per weight of D-galacturonic acid natural polymer derivative was prepared by adding water to the polymer, followed by dropwise addition of an 5 M aqueous basic solution with continuous stirring and keeping the temperature of the mixture from 0 to 50° C. until the polymer is totally dissolved.

A soap solution is prepared by dissolving 13 grams of soap in 50 ml of water with continuous stirring. The solution obtained has a pH near 9.7.

Subsequently, 10 ml of the soapy aqueous solution were added to 15 ml of the aqueous polymer-containing solution. The addition is done while keeping the temperature from 0-50° C. degrees and with continuous stirring. The obtained solution has a pH near 9.6 and it was adjusted to near 9.75 by addition of base. To the resulting solution it was added 0.4 ml of glycerin, colorants and fragrances. The resulting mixture was extended over an anti-adhesive surface and allowed to dry. Once dried, it was cut in thin films.

### EXAMPLE 2

15 ml of an aqueous solution containing 8% per weight of the D-galacturonic acid natural polymer derivative was prepared as described in Example 1 and followed by the addition of 0.4 ml of glycerin.

An aqueous 50 ml solution containing 13 grams of soap was prepared as described in Example 1. To the obtained solution were added colorants and fragrance. The obtained solution has a pH near 9.6.

Subsequently, 10 ml of the soapy aqueous solution were added to 15 ml of the aqueous polymer-containing solution. The addition is done while keeping the temperature from 0-50° C. degrees and with continuous stirring. The pH of the resulting solution was adjusted to 9.8 by addition of a base. The resulting mixture was extended over an anti-adhesive surface and allowed to dry. Once dried, it was cut in thin films.

I claim:

1. A film-shape soap no containing synthetic polymers, comprising (a) a natural polymer derivative of D-galacturonic acid that is lineal and partially esterified with methyl groups and that has a molecular weight from 20,000 to 400,000 and (b) a soap.

2. The film-shape soap of claim 1, wherein the soap in (b) is prepared from one or more of the acids selected from the group comprising aranquinodic, esteric, lauric, linoleic, linolenic, oleic, palmitic and myristic acids.

3. The film-shape soap of claim 1, further comprising additives selected from one or more of the group comprising fragrances, color agents, moisturizers, antioxidants and antibacterial agents.

4. The film-shape soap of claim 1, wherein the said film has a thickness of from about five micrometers to one millimeter.

5. The film-shape soap of claim 4, wherein the said film has a thickness of from about 20 micrometers to 300 micrometers.

6. A process for the preparation of the film shape soap of claim 1, comprising:
a) preparing an aqueous solution of soap;
b) preparing an aqueous solution of the polymer derivative of D-galacturonic acid;
c) mixing the said aqueous solutions; and
d) allowing the mixture to dry out over an extended flat surface.

7) The process of claim 6, wherein any of the aqueous solutions described in (a) or (b) contain one or more of the soap additives selected from fragrances, color agents, moisturizers, antioxidants and antibacterial agents.

8) The process of claim 6, wherein to the mixture obtained in step C) contains one or more of the soap additives selected from fragrances, color agents, moisturizers, antioxidants and antibacterial agents.
